(19) United States
(12) Patent Application Publication
(10) Pub. No.: US 2008/0039913 A1
(43) Pub. Date: Feb. 14, 2008

(54) Aromatherapy blanket pack

(76) Inventor: Hagay Mizrahi, Woodland Hills, CA (US)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 11/501,355

(22) Filed: Aug. 9, 2006

(51) Int. Cl. A61F 7/00

(52) U.S. Cl. 607/114

(57) ABSTRACT

An aromatherapy blanket includes a flexible, aroma-permeable cover that has upper and lower layers. The upper and lower layers are joined at their outer edges and define a space between them. The space is divided into a number of subspaces which are filled with a quantity of thermal reservoir material and a quantity of aromatherapy material. The typically tube-shaped subspaces are spaced apart from one another by a first predetermined distance. The subspaces are divided into substantially equal portions to prevent shifting of the thermal material and the aromatherapy material. The thermal reservoir materials are selected from the group consisting of corn, rice, wheat, oats, barley, beans and flaxseed. The aromatherapy materials are selected from the group consisting of cinnamon, chamomile, lemongrass, peppermint, rosemary, spearmint, valerian root, white willow, yarrow, lavender ginger and yellow dock root. The blanket includes a washable containment cover sized and shaped to enclose the blanket.

### FIELD OF INVENTION

The invention pertains to devices for alleviating muscle soreness, fatigue and stress. More particularly, the invention relates to herb packs that can be heated or cooled and then applied to the body to provide relief through temperature difference and aromatherapy related to the herbs contained in a light-weight blanket pack.

### BACKGROUND OF THE INVENTION

Heating and cooling pads have long been in use for alleviating soreness, stiffness, aching muscles and the results of various types of injuries. Likewise, aromatherapy products have been in use in Eastern cultures for hundreds of years and are now achieving prominence in the West. The combination of heating and cooling devices with aromatherapy products has served to provide increased benefits for those with muscle, tendon and joint problems. A number of inventions have been developed that use one or more of these components to alleviate pain or soreness.

U.S. Pat. No. 4,231,125, issued to Tittl, discloses a beach towel or the like for use with a pillow, and characterized by simplicity and economy of construction, and the use of elements substantially impervious to corrosion by salt and damage from sand. The beach towel comprises an elongate, substantially rectangular fabric piece defining opposing, laterally extending top and bottom end edges and opposing, longitudinally extending side edges. A pair of longitudinally spaced-apart and laterally extending attachment members are secured to the fabric piece proximate its top end for permitting the top end of the fabric piece to be folded over itself and releasably attached in such position to form a tube for enclosing a pillow. One of the attachment members comprises a patch having numerous rows of hook-like projections, and the other of the pair of attachment members comprises a patch of loose, fibrous, non-woven material for releasably interlocking with the hook-like projections.

U.S. Pat. No. 5,819,347, issued to Masuda describes a pillow comprising at least three substantially flat bags successively layered, each of intermediate layer bags other than top and bottom layer bags is provided with closure means and provided substantially at the middle of each intermediate layer bag with a pocket having also its own closure means through which cedar chips are enclosed in this pocket. Chips of synthetic resin are enclosed in one of the outermost layer bags and a fibrous member containing aromatic essence of Japanese cypress is enclosed in the other outermost layer bag. No content is enclosed in the bags other than the intermediate layer bags in their regions over- and underlying the pockets so that a recess having no significant thickness is formed in those regions over- and underlying the pockets. The regions of the intermediate layer bag(s) extending adjacent the periphery of the pillow may be loaded with cedar chip bags so as to adjust a thickness of the pillow in its peripheral region. In this manner, the novel pillow supports the user's head and neck with comfortable stability and contributes to elimination of minute forms of life such as ticks as well as to a desired aroma-therapeutic effect.

U.S. Pat. No. 4,586,506, issued to Nangle discloses an elastic wrap which is to be utilized in connection with a flexible walled container. The flexible walled container includes an access door within which is located either a heat or cold pack. Pad type of attaching means is to be located at a desired location on the elastic wrap which is to connect with a similar type of pad mounted on the container to secure the container to the elastic wrap. U.S. Patent Application No. 2002/0198580, published by Clayton describes a therapeutic wrap designed to be applied to various portions of an individual, such as the neck and shoulders. The wrap contains a plurality of narrow channels into which a filler material, such as Basmati rice, is provided. Various herbs such as chamomile or lavender are also provided within each of the channels to allow for aromatherapy.

U.S. Pat. No. 6,699,271, issued to Clayton, describes a therapeutic wrap designed to be applied to various portions of an individual, such as the neck and shoulders. The wrap contains a plurality of narrow channels into which a filler material, such as Basmati rice, is provided. Various herbs such as chamomile or lavender are also provided within each of the channels to allow for aromatherapy. The rice serves as heat-transferring material. Each channel is separated from its adjoining channel by stitching it closed such. The neck wrap is designed to encircle the neck of the user and fasten with Velcro fasteners. The Velcro fasteners and the use of narrow channels keep the filler material within the wrap from shifting and hold it close to the neck during treatment. Basmati rice and aromatic herbs are the preferred fillers. In use, the wrap is heated by placing it in a conventional oven, crockpot or microwave, or cooled by placing it into a freezer or refrigerator.

U.S. Pat. No. 5,476,492, issued to Unrug, discloses a body warmer for therapeutic purposes containing whole herb seed. The body warmer is comprised of a shell of an elastic breathable and pliable material, divided into chambers containing rebufacient herbal material in the form of whole seed grains, and means of attachment to keep the warmer in place in a desired area of the human body. The shell is made in two layers of fabric fastened along two long sides at side seams. Other seams divide the shell into a number of tubular chambers filled with herb seed. Ribbon tie straps attached to corners of the shell are used to fasten the shoulder warmer pad to the body.

U.S. Pat. No. 5,603,727, issued to Clark et al., is directed to a thermal pack having temperature-retaining particles within the packet. A plurality of barriers located within the packet form migration paths and retaining areas. The pack that has pliable container consisting of top and bottom surfaces connected about their periphery. Barriers within the compartment to direct the migration of the heat retaining particles in the compartment are formed by sewing portions of the top and bottom surfaces together in a chevron shape. Heat retaining particles are preferably re-cleaned animal feed corn, which can be scented. The thermal material can be either heated or cooled depending on the appropriate treatment.

U.S. Pat. No. 5,948,010, issued to Adamee, discloses a therapeutic heat application device. The device comprises a pad portion having a wide central portion divided into a plurality of narrow sections by vertically extending seams. Each of the tapered side portions are divided into two sections by a horizontally extending seam. The sections of the central portion and side portions are filled with a granular natural filler material where the seams would maintain the proper positioning of the filler material throughout the pad portion. A pair of straps have hook and loop fasteners for fastening the pad around the body of the wearer. In use, the device would be heated in a microwave oven or chilled for cold therapy.

U.S. Pat. No. 5,890,487, issued to Kimmel, is directed to a flat fabric bag that holds a quantity of dried Indian corn, which can be scented. The bag can then be heated in a microwave oven or cooled in a freezer. The heating pad that is formed by sewing two pieces of cloth together to form closed bag which is then filled with corn. To keep the corn spread throughout the bag, a number of baffle stitches are used which divide the bag into sections that hold a uniform quantity of corn. The bag is used primarily over the shoulders, where the wide end portions drape over the tops of the shoulders and the long center portion runs along the upper back or neck.

It is an objective of the present invention to provide a means to provide a means to alleviate muscle soreness, cramps, aches and pains, etc. without the use of drugs or invasive procedures. It is a further objective to provide a lightweight blanket that provides these benefits that can be comfortably and easily handled by a user. It is a still further objective of the invention to provide a blanket that can provide both heating and cooling to the body. It is yet a further objective to provide a blanket that includes the benefits of aromatherapy for the user. Finally, it is an objective of the invention to provide a blanket with the above benefits that fits within a removable, washable cover.

While some of the objectives of the present invention are disclosed in the prior art, none of the inventions found include all of the requirements identified.

### SUMMARY OF THE INVENTION

The present invention addresses all of the deficiencies of heating and cooling blanket inventions and satisfies all of the objectives described above.

(1) An aromatherapy blanket providing the desired features includes a flexible, aroma-permeable cover. The cover has an upper layer and a lower layer. The upper and lower layers are joined at their outer edges and define a space between them. The space is divided into a plurality of subspaces. A quantity of thermal reservoir material is provided. A quantity of aromatherapy material is provided. The thermal material and the aromatherapy material are secured within the subspaces. The subspaces are spaced apart from one another by a first predetermined distance. When blanket is heated or cooled it is placed on the body of the user, thereby heating or cooling the user while providing aromatherapy benefits.

(2) In a variant of the invention, the subspaces are a series of tube-shaped containers.

(3) In another variant, the tube-shaped containers are divided into substantially equal portions to prevent shifting of the thermal material and the aromatherapy material.

(4) In yet another variant, a washable containment cover is provided. The cover is sized and shaped to enclose the blanket.

(5) In still another variant, fasteners for attaching the blanket to the containment cover are provided.

(6) In a further variant, the fasteners are selected from the group consisting of button, hook and loop fastener, zipper and tie string.

(7) In yet a further variant, a closure means for closing an open end of the containment cover is provided.

(8) In still a further variant, the closure means is selected from the group consisting of button, hook and loop fastener, zipper and tie string.

(9) In another variant of the invention, the thermal reservoir materials are selected from the group consisting of corn, rice, wheat, oats, barley, beans and flaxseed.

(10) In still another variant, the aromatherapy materials are selected from the group consisting of cinnamon, chamomile, lemongrass, peppermint, rosemary, spearmint, valerian root, white willow, yarrow, lavender ginger and yellow dock root.

(11) In yet another variant, the subspaces are closed with stitching.

(12) In a further variant, the subspaces have openable closures to permit introduction of alternative thermal and aromatherapy materials.

(13) In a final variant, the openable closures are selected from the group consisting of hook and loop fastener, zipper, button and tie string.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a perspective view of the preferred embodiment of the invention illustrating the subspaces containing the thermal and aromatherapy materials;

FIG. 2 is a partial cross-sectional view of the FIG. 1 embodiment taken along the line 2-2;

FIG. 3 is a perspective view of a washable containment cover for the aromatherapy blanket; and

FIG. 4 is a a perspective view of of the FIG. 1 embodiment partially inserted in the containment cover.

### DETAILED DESCRIPTION OF THE PREFERRED EMBODIMENT

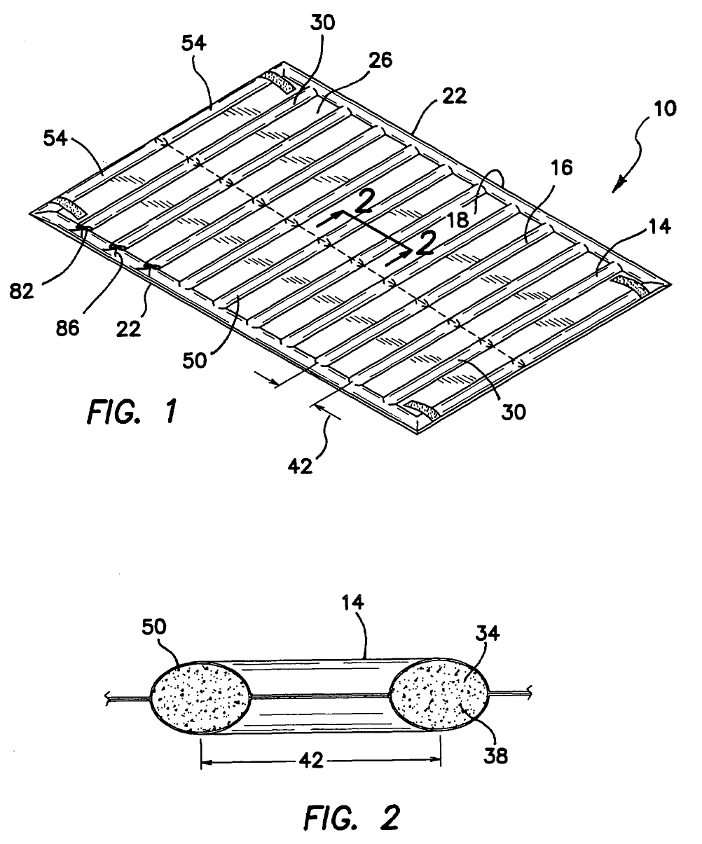

(1) FIGS. 1-4 illustrate an aromatherapy blanket 10 providing the desired features that includes a flexible, aroma-permeable cover 14. The cover 14 has an upper layer 16 and a lower layer 18. The upper 16 and lower 18 layers are joined at their outer edges 22 and define a space 26 between them. The space 26 is divided into a plurality of subspaces 30. As illustrated in FIG. 2, a quantity of thermal reservoir material 34 is provided. A quantity of aromatherapy material 38 is provided. The thermal material 34 and the aromatherapy material 38 are secured within the subspaces 30. The subspaces 30 are spaced apart from one another by a first predetermined distance 42. When blanket 10 is heated or cooled it is placed on the body of the user 46, thereby heating or cooling the user 46 while providing aromatherapy benefits.

(2) In a variant of the invention, the subspaces 30 are a series of tube-shaped containers 50.

(3) In another variant, as illustrated in FIG. 1, the tube-shaped containers 50 are divided into substantially equal portions 54 to prevent shifting of the thermal material 34 and the aromatherapy material 38.

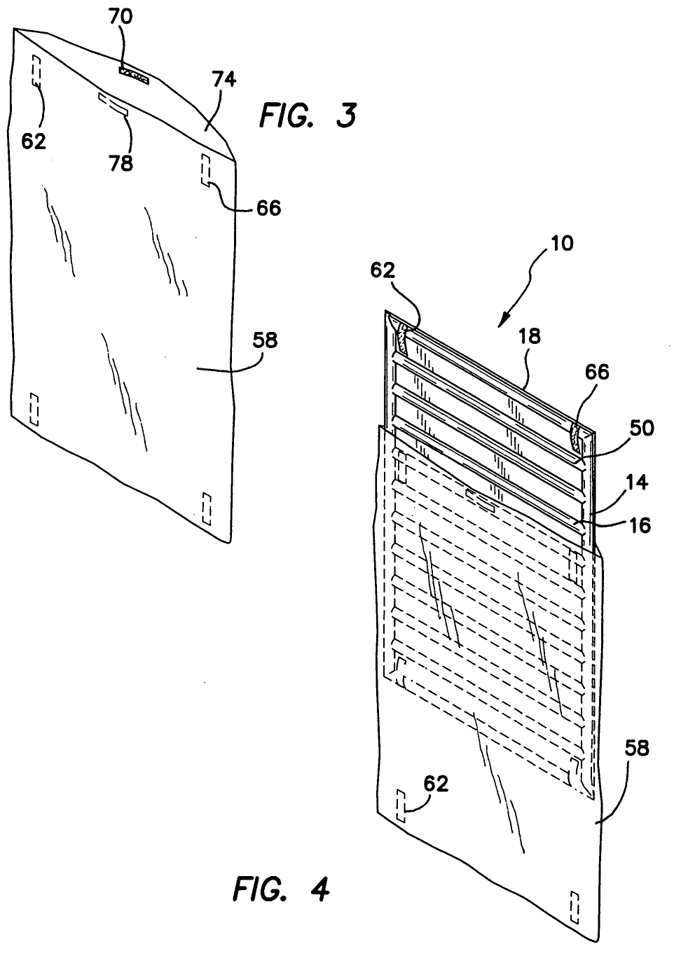

(4) In yet another variant, as illustrated in FIGS. 3 and 4, a washable containment cover 58 is provided. The cover 58 is sized and shaped to enclose the blanket 10.

(5) In still another variant, fasteners 62 for attaching the blanket 10 to the containment cover 58 are provided.

(6) In a further variant, the fasteners 62 are selected from the group consisting of button (not shown), hook and loop fastener 66, zipper (not shown) and tie string (not shown).

(7) In yet a further variant, a closure means 70 for closing an open end 74 of the containment cover 58 is provided.

(8) In still a further variant, the closure means 70 is selected from the group consisting of button (not shown), hook and loop fastener 78, zipper (not shown) and tie string (not shown).

(9) In another variant of the invention, the thermal reservoir materials 34 are selected from the group consisting of corn, rice, wheat, oats, barley, beans and flaxseed.

(10) In still another variant, the aromatherapy materials 38 are selected from the group consisting of cinnamon, chamomile, lemongrass, peppermint, rosemary, spearmint, valerian root, white willow, yarrow, lavender ginger and yellow dock root.

(11) In yet another variant, the subspaces 30 are closed with stitching.

(12) In a further variant, the subspaces 30 have openable closures 82 to permit introduction of alternative thermal 34 and aromatherapy 38 materials.

(13) In a final variant, the openable closures 82 are selected from the group consisting of hook and loop fastener (not shown), zipper (not shown), button (not shown) and tie string 86.

The aromatherapy blanket 10 has been described with reference to particular embodiments. Other modifications and enhancements can be made without departing from the spirit and scope of the claims that follow.

1. An aromatherapy blanket, comprising:
a flexible, aroma-permeable cover, said cover having an upper layer and a lower layer, said upper and lower layers being joined at outer edges thereof and defining a space therebetween, said space being divided into a plurality of subspaces;
a quantity of thermal reservoir material;
a quantity of aromatherapy material;
said thermal material and said aromatherapy material being secured within said subspaces;
said subspaces being spaced apart from one another by a first predetermined distance; and
whereby, when said herb pack is heated or cooled it is placed on the body of said user, thereby heating or cooling said user while providing aromatherapy benefits.

2. The aromatherapy blanket, as described in claim 1, wherein said subspaces are a series of tube-shaped containers.

3. The aromatherapy blanket, as described in claim 2, wherein said tube-shaped containers are divided into substantially equal portions to prevent shifting of said thermal material and said aromatherapy material.

4. The aromatherapy blanket, as described in claim 1, further comprising a washable containment cover, said cover being sized and shaped to enclose said blanket.

5. The aromatherapy blanket, as described in claim 4, further comprising fasteners for attaching said blanket to said containment cover.

6. The aromatherapy blanket, as described in claim 5, wherein said fasteners are selected from the group consisting of:
button, hook and loop fastener, zipper and tie string.

7. The aromatherapy blanket, as described in claim 4, further comprising a closure means for closing an open end of said containment cover.

8. The aromatherapy blanket, as described in claim 7, wherein said closure means are selected from the group consisting of:
button, hook and loop fastener, zipper and tie string.

9. The aromatherapy blanket, as described in claim 1, wherein said thermal reservoir materials are selected from the group consisting of:
corn, rice, wheat, oats, barley, beans and flaxseed.

10. The aromatherapy blanket, as described in claim 1, wherein said aromatherapy materials are selected from the group consisting of:
cinnamon, chamomile, lemongrass, peppermint, rosemary, spearmint, valerian root, white willow, yarrow, lavender ginger and yellow dock root.

11. The aromatherapy blanket, as described in claim 1, wherein said subspaces are closed with stitching.

12. The aromatherapy blanket, as described in claim 1, wherein said subspaces have openable closures to permit introduction of alternative thermal and aromatherapy materials.

13. The aromatherapy blanket, as described in claim 12, wherein said openable closures are selected from the group consisting of:
hook and loop fastener, zipper, button and tie string.
